(19) United States
(12) Patent Application Publication
(10) Pub. No.: US 2015/0038872 A1
(43) Pub. Date: Feb. 5, 2015

(54) MULTIPLE-ELECTRODE ELECTRICAL IMPEDANCE SENSING BIOPSY SAMPLING DEVICE AND METHOD

(71) Applicant: The Trustees of Dartmouth College, Hanover, NH (US)

(72) Inventor: Ryan Halter, Orford, NH (US)

(73) Assignee: The Trustees of Dartmouth College, Hanover, NH (US)

(21) Appl. No.: 13/958,121

(22) Filed: Aug. 2, 2013

(51) Int. Cl. A61B 5/06; A61B 5/053; A61B 10/04; A61B 10/02

(52) U.S. Cl. CPC A61B 5/063; A61B 10/0275; A61B 5/0538; A61B 10/04; A61B 2010/045
USPC 600/547

(57) ABSTRACT

A biopsy sampling device has an inner trocar having a sampling opening, an outer needle with 4 or more electrodes on it, an impedance measuring apparatus coupled to drive current through a first and a second electrode, while measuring voltages through a third and fourth electrodes to measure an impedance of tissue adjacent to the electrodes, the inner trocar slideably engaged within the central cavity of the outer needle such that its sharpened tip and sampling opening can protrude from an end of the outer needle and be removed to capture a sample in the sampling opening. The device is used to obtain biopsy samples by advancing the device into an organ while observing impedance of tissue near or adjacent the device before the operator withdraws the trocar to obtain a biopsy sample of an inclusion in the organ.

### GOVERNMENT RIGHTS

The present apparatus was developed with the aid US Department of Defense Congressionally Directed Medical Research Program grant W81XH-07-1-0104. The United States Government has certain rights in the herein described apparatus.

### FIELD

The present apparatus relates to the field of tissue sampling devices for obtaining specimens of tissue for pathological examination, and to the field of medical instrumentation devices.

### BACKGROUND

When lumps, tumors, inhomogenicities, or other inclusions appear within human and other biological tissues, imaging is often insufficient to fully identify and type the inclusion. For example, imaging alone cannot provide genetic analysis of a tumor, such as may be useful in determining susceptibility to particular chemotherapy agents. It is therefore often desirable to obtain samples of the inclusion for analysis so as to determine a type and treatment susceptibility of the inclusion. Typing of inclusions is also desirable to assist in determining whether treatment is necessary; since some inclusions may be malignant, others benign, and others may be abscesses or cysts. Cysts and abscesses require quite different treatment from malignant inclusions.

Samples are often taken from inclusions using a sampling device having an outer tube and an inner probe or needle having a cutting cavity on a side. The device is inserted into the inclusion and the inner probe or needle is operated to capture a small piece of tissue from the inclusion in the cavity. The device is removed and the sample analyzed.

A problem when taking samples of inclusions, especially smaller inclusions, in tissues is that it can be difficult to ensure that the sample is taken of the inclusion and not of adjacent, likely healthy, tissue. When normal, nearby, tissue is sampled instead of the inclusion, pathological analysis of the sample will not give a correct diagnosis and may give sufficiently misleading information that no or inappropriate treatment is provided to patients instead of appropriate curative treatment. Similarly, even when a tumor is sampled, current sampling devices may capture small samples not representative of tumor as a whole, also potentially leading to inappropriate treatment. For example, a single sample might be taken from a necrotic core of a tumor, while omitting better-vascularized and rapidly-growing peripheral tissue.

In order to obtain samples from an inclusion instead of from normal tissue, imaging-guided biopsy techniques may be used. For example, Computed Tomography (CT)-guided biopsy techniques are often used with some organs. These techniques require taking multiple images of a patient to observe both the inclusion and a sampling device; the images are taken at intervals during the process of inserting and manipulating the sampling device into the inclusion. CT-guided biopsy techniques pose issues with high radiation dose from multiple CT images, and do not always provide good resolution of the inclusions, especially when the inclusions are in low density tissues surrounded by high density tissues. Further, CT machines are somewhat bulky and moderately expensive. Alternatively, Magnetic Resonance Imaging (MRI) may be used to image tissue immobilized in a frame, and the frame and images used to guide sampling. MRI machines, however, are even more expensive than CT machines, cannot be used on some patients due to metallic implants, and both tissue and inclusion may shift as a biopsy sampling device is inserted into the tissue.

WO/2002/085216 describes a biopsy sampling device adapted for Magnetic Resonance Imaging (MRI)-guided biopsies. This device has an outer shield and an inner probe, where the inner probe is electrically insulated from the outer shield by an insulation layer on the inner conductor. This device serves as a radio-frequency antenna to sense resonance during operation of an MRI system, requiring an expensive MRI machine during taking of a biopsy sample. Also, since it is intended for use within the intense magnetic field of an MRI system, it must be made of non-ferrous materials that are not affected by, and do not affect, the magnetic field of the MRI machine. This device is used to sample an organ while imaging the organ, so that samples may be obtained from particular suspicious inclusions within the organ.

It is desirable to find alternative ways of guiding biopsy sampling devices to obtain samples of tumors and other inclusions in organs; in particular it is desirable to find ways that do not require use of such an expensive and bulky device as an MRI imaging system while obtaining biopsy samples for pathological analysis. It is also desirable to sense the pathological state of the tissue in areas close to where the sample was collected to provide a more accurate estimate of disease extent, if any.

We have previously proposed a two-electrode electrical impedance imaging device.

### SUMMARY

In an embodiment, a biopsy sampling device has an inner trocar having a sharpened tip with a sampling opening, an outer needle having a central cavity within which the trocar slides, the outer needle formed of a material selected from the group consisting of insulators and metal coated with an insulator, at least a first, a second, a third, and a fourth electrical conductor formed on the outer needle, the conductors forming a first, a second, a third, and a fourth electrode, an insulating coating formed over at least a central portion of the electrical conductors, an impedance measuring apparatus coupled to drive current through a first and a second selected electrode of the electrodes, and to measure voltages through a third and fourth selected electrode of the electrodes to measure an impedance of tissue adjacent to the electrodes, the inner trocar slideably engaged within the central cavity of the outer needle such that its sharpened tip and sampling opening can protrude from an end of the outer needle; wherein the inner trocar is adapted to be removed from the outer needle, thereby capturing a sample in the sampling opening.

In another embodiment, a method of obtaining biopsy samples from a subject includes advancing a sampling device into an organ while monitoring impedance characteristics between electrodes formed on an exterior of an outer needle of the sampling device, the outer needle of the sampling device having more than two electrodes; displaying to an operator on a display at least one a first impedance characteristic of organ tissue adjacent the electrodes, the impedance characteristic determined by driving current through at least one pair of the electrodes while measuring voltages between a second pair of the electrodes, and the impedance characteristic on the display is updated regularly; and upon observing a change of impedance, the operator withdrawing the central trocar of the sampling device to obtain a biopsy sample of an inclusion in the organ.

### BRIEF DESCRIPTION OF THE DRAWINGS

FIG. 1 is a sectional diagram of a tip of a prior biopsy sampling device with insulating coatings.

FIG. 2 is a block diagram of our prior proposal for a two-electrode electrical-impedance-guided biopsy sampling device.

FIG. 3 is a profile view of an electrical-impedance guided biopsy sampling device having four electrodes.

FIG. 4 is a cross sectional view of an electrical-impedance guided biopsy sampling device having eight electrodes.

FIG. 5 is a cross sectional view of the electrical-impedance guided sampling device of FIG. 3 taken at A-A in FIG. 3.

FIG. 6 is a cross-sectional view of the electrical-impedance guided sampling device of FIG. 3 taken at B-B in FIG. 3.

FIGS. 7A, 7B, 7C, and 7D illustrate alternative connections of the voltage measurement and current driver circuitry through the switching matrix of the embodiment of FIG. 3.

FIG. 8 is a flow chart of one method of operating the sampling device wherein the device is used to obtain impedance information at sample points within an organ.

FIG. 9 is a flow chart of a method of operating the sampling device wherein the device is used to guide acquisition of samples while advancing the sampling device to desired sample locations.

FIG. 10 is a flowchart illustrating a method of using the biopsy sampling device herein described to obtain samples of a lesion.

FIG. 11 is an illustration of making multiple voltage measurements simultaneously using a biopsy sampler having more than four electrodes.

FIG. 12 illustrates an alternative electrode arrangement for the outer needle of the multiple-electrode sampling device.

FIG. 13 illustrates another alternative electrode arrangement for the outer needle of the multiple-electrode sampling device.

FIG. 14 illustrates another alternative electrode arrangement for the outer needle of the multiple-electrode sampling device having multiple layers of electrodes.

### DETAILED DESCRIPTION OF THE EMBODIMENTS

In a study of radical pro statectomy specimens (Halter R J, Schned A R, Heaney J A, et al. Electrical impedance spectroscopy of benign and malignant pro static tissues. Journal of Urology, 179(4):1580-1586, 2008) from fourteen men, it was found that some tumors of the prostrate have an electrical impedance (inverse of admittance) that differs from the electrical properties of surrounding, normal, tissues. In particular, at least some adenocarcinoma (malignant) tumors of the prostate were found to have electrical conductivity and permittivity (components of electrical impedance) that differed from tissues associated with benign prostate hypertrophy or normal prostate stroma at frequencies of greater than 92 KHz. Particular samples of adenocarcinoma of the prostate were found to have significantly lower conductivity (higher resistance) than normal prostate stroma.

A more recent study (Halter R J, Schned A R, Heaney J A, et al. Electrical properties of prostatic tissues: I. Single frequency admittivity properties. Journal of Urology, 182:1600-1607, 2009) has also been done. In this study of tissue samples of adenocarcinoma, benign pro static hyperplasia, non-hyperplastic glandular tissue, and stroma samples taken from radical pro statectomy specimens from 50 men, it was shown that, in addition to significant conductivity differences between malignant and benign prostate tissue, there are significant permittivity differences. The direction and magnitude of these differences changes depending on the frequency at which the electrical properties were gauged. In particular, the permittivity of prostate cancer at 100 kHz is twice that of benign pro static hyperplasia, non-hyperplastic glandular tissue, and normal pro static stroma. When permittivity at 100 kHz was used to discriminate cancer from benign tissues it provided a specificity of 77% at a sensitivity level of 70%.

The electrical properties of tissue are a function of the AC frequency at which they are sampled. This frequency dependence is also a function of tissue morphology and this spectral dependence has the potential to provide enhanced clinical utility. In this same cohort of 50 men, the electrical properties were sampled at 31 logarithmically spaced frequencies ranging from 100 Hz to 100 kHz. Four multi-frequency based spectral parameters defining the recorded spectrum (τ_{∞}, Δσ, f_{c}, and α) using a Cole-type model were extracted from each of the electrical property spectra. The Cole-type model is similar to that described in Cole K S and Cole R H, Dispersion and absorption in dielectrics: I. Alternating current characteristics J Chem Phys, 9: 341-351, 1941. These spectral parameters are typically thought to represent:
1) σ_{∞}, (extrapolated impedance at infinite frequency): a measure of cumulative intra- and extra-cellular fluid conductivity
2) Δσ (difference between extrapolated impedance at zero frequency and at infinite frequency): a measure of the intra- and extra-cellular volume
3) f_{c} (a relaxation frequency, derived as an inverse of a relaxation time): a measure of cell membrane quantity and viability
4) α (a measure of a broadness of the spectra): a measure of tissue heterogeneity

The results of the spectral decomposition are presented in Halter R J, Schned A R, Heaney J A, et al. Electrical properties of prostatic tissues: II. Spectral admittivity properties Journal of Urology, 182:1608-1613, 2009. Significant differences between malignant and benign prostate were noted for σ_{∞}, Δσ, and f_{c}. Of the spectral parameters, f_{c} provided the best cancer discrimination with a specificity of 81.5% at a sensitivity level of 70%. Spectral representations other than the Cole-model can be employed to parameterize the frequency-dependent electrical properties. These spectral parameters provide more contrast than the discrete frequency parameters (conductivity and permittivity), but require a longer acquisition time since the electrical properties at multiple frequencies must be sampled. Depending on the clinical situation either spectral or discrete frequency electrical properties could be gauged.

Finally, in Halter R J, Schned A R, Heaney J A, Hartov A. Passive bioelectrical properties for assessing high- and low-grade prostate adenocarcinoma. The Prostate, 71:1759-1767, 2011 it is shown that these electrical properties (both discrete frequency and spectral) provide enhanced discriminatory power when just high-grade prostate cancers were compared to all benign tissues. Specifically, of the 546 prostate tissue samples explored in the study, 71 were identified as cancer and 465 as benign. ∈ (at 100 kHz), Δσ, σ_{∞}, and f_{c} provided the most discriminatory power with area under the curves (AUCs) ranging from 0.77-0.82 for detecting any cancer, 0.72-0.8 for low-grade cancer, and increasing to 0.87-0.9 for detecting high-grade cancer. Further, ∈ (at 100 kHz), Δσ, and σ_{∞}, provided AUCs ranging from 0.74 to 0.75 for discriminating between low- and high-grade cancers.

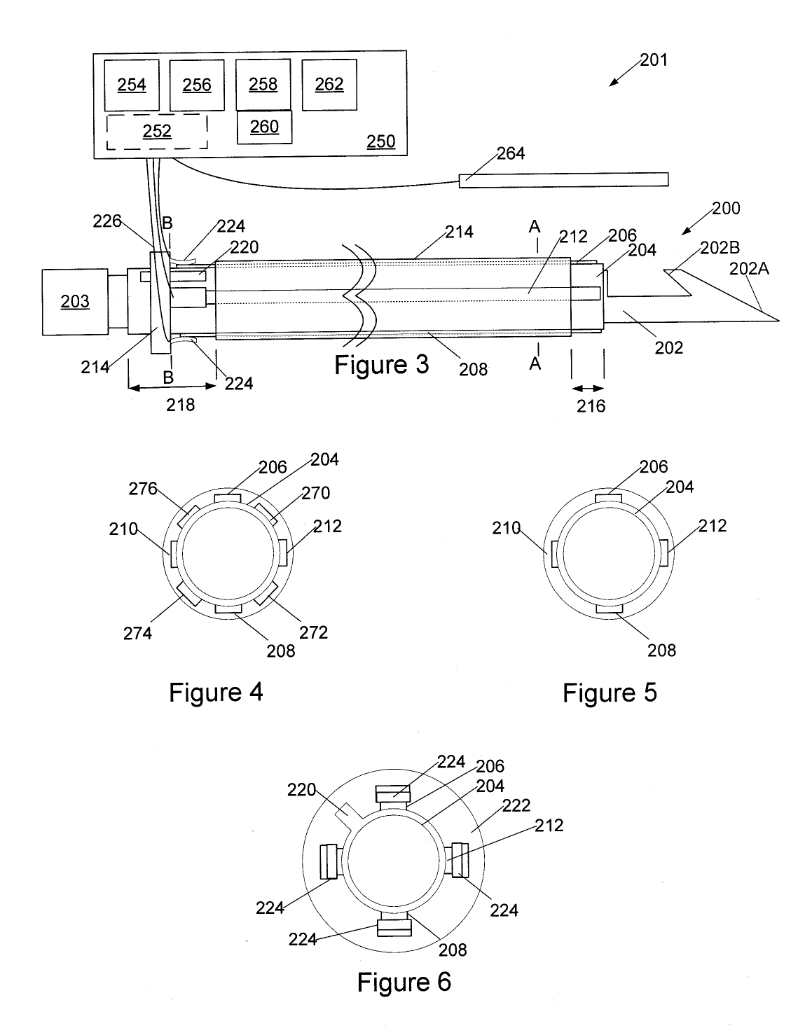

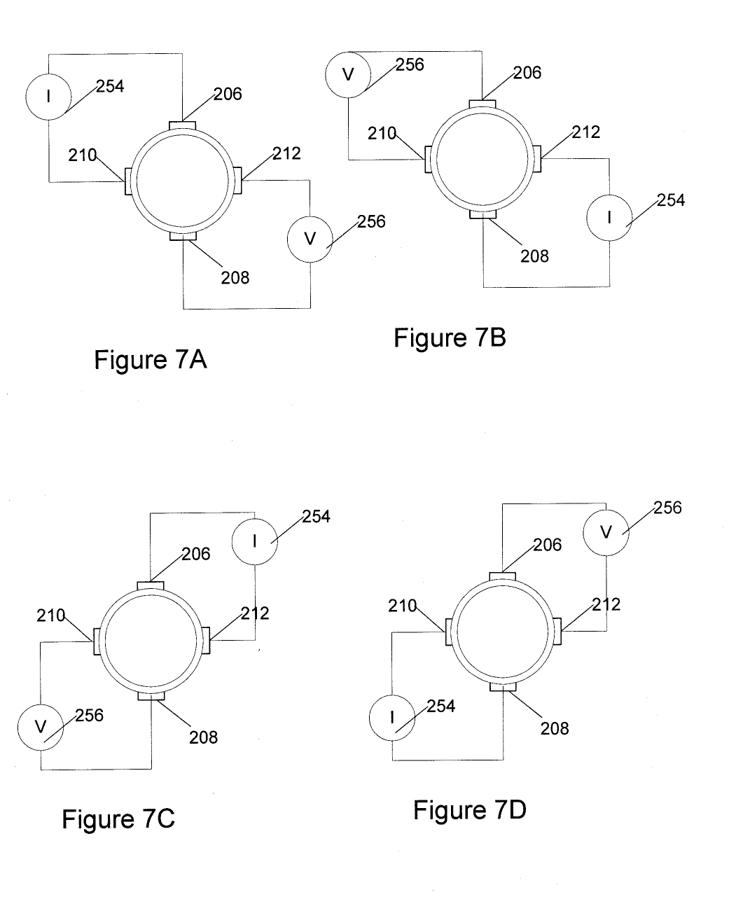

Similar electrical property differences have been noted between malignant and benign tissues in a number of other organs including breast, liver, kidney, and others. The sampling device herein described, including the improved multiple-electrode sampling device of FIGS. 3-12, my prove useful in evaluating and sampling tumors in these organs.

It is believed that electrical conductivity and permittivity measurements, and the Cole-Cole spectral parameters derived from them, made using a biopsy sampling device as an electrode will provide information about tissue near the sampling device at the time a sample is taken, and may be able to provide some guidance to a physician so that samples may be taken of malignant inclusions as well as surrounding tissues. The physician may insert the device into tissue while observing impedance, and take samples when the sampling device has penetrated an inclusion having impedance differing from that of most surrounding tissue.

These conductivity and permittivity measurements may also provide some additional diagnostic information regarding the extent of disease since many biopsy samples only show a small foci of cancer. These measurements may indicate if the tissue surrounding the biopsy site is diseased or not.

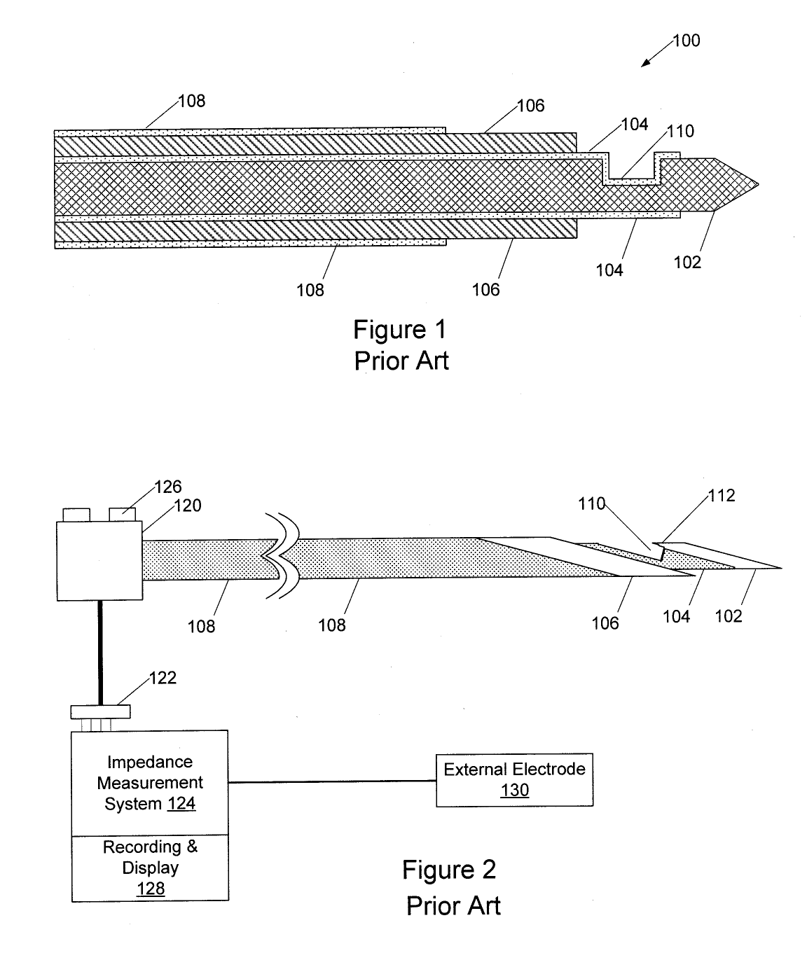

Our earlier biopsy sampling device 100 (FIGS. 1 and 2) has a central sampling needle trocar 102, which may have a single-tapered (as shown in FIG. 2) or a double-tapered (as shown in FIG. 1) sharpened tip. The central sampling needle trocar 102 has an electrically insulating, biocompatible, coating 104 adherent thereto, such as polyimide or Epoxylite® 6000 M. Epoxylite is a trademark of Elantus PDG Inc, St Lois, MO, a subsidiary of Elantas of Wessel, Germany.

The central sampling needle trocar 102 and its coating 104 is slideably engaged within an outer hollow needle 106. In an embodiment, outer hollow needle 106 is an 18-gauge needle. Similarly, all but a tip portion of outer hollow needle 106 is coated with an outer-needle insulating, biocompatible, coating 108 adherent thereto; in an embodiment this is formed of the same polyimide or Epoxylite 6000 M material used for the coating on the sampling needle trocar 102. In an embodiment, the uninsulated tip portion of the outer needle has length about two millimeters.

In an embodiment, insulating coating 104 is less than fifty microns thick so that the central sampling needle trocar 102 of about ninety-nine hundredths inch diameter can freely slide within the outer hollow needle 106.

Since the sampling device 100 need not be used in a magnetic resonance imaging environment, in an embodiment the central sampling needle trocar 102 and outer hollow needle 106 are made of ferrous metal, such as stainless steel as known in the surgical instrument art.

Central sampling needle trocar 102 has a sample slot 110 cut into it. When the device is inserted into tissue with the sampling needle trocar fully extended, tissue—possibly including a portion of an inclusion—enters the sample slot 110. The sampling needle trocar 102 may then be withdrawn through the outer needle 106 and a cutting edge 112 separates a sample of the tissue from the tissue. The sample may be placed in a pathology sample container (not shown) and the sampling needle trocar 102 reinserted into the outer needle 106 to obtain additional samples.

Outer hollow needle 106 is fitted with a manipulation handle 120, which is adapted with mechanical keying apparatus such that, in embodiments like that of FIG. 2 with a single-tapered tip, sampling needle trocar 102 is not free to rotate with respect to outer hollow needle 106. Four wires are brought out to a connector 122 from the needles 102, 106, two attached to the sampling needle trocar 102 and two to the outer needle 106. One wire attached to sampling needle trocar 102 is coupled through connector 122 to a stimulus circuit of impedance measuring system 124, the other wire connected to sampling needle trocar 102 is coupled to a measurement circuit of impedance measuring system 124. Similarly, one wire attached to outer needle 106 is coupled through connector 122 to a stimulus circuit of impedance measuring system 124, the other wire connected to outer needle 106 is coupled to a measurement circuit of impedance measuring system 124 having display and recording apparatus 128. In an alternative embodiment, more subject to interference by dirty or loose connections, only two electrical connections are used, one to the trocar 102 and one to the outer needle 106. In an alternative embodiment, there is also one or more external electrodes 130 for contacting a surface of a subject.

The manipulation handle 120 is also fitted with an impedance test button 126 to trigger measurement and acquisition of electrical impedance data.

For some inclusions and tissues, the sampling device of FIGS. 1 and 2 is not sufficiently accurate. For example, impedance measured by the device of FIGS. 1 and 2 is a sum of tissue impedance and a contact impedance where each electrode, such as bare portion of outer needle 106 and bare portion of trocar 102, contacts tissue. Further, the device is not able to determine which side of the inclusion the sampling device may be located on. It is believed that accuracy of sensing tissue impedance can be enhanced by changing to a multiple-electrode technique where stimulus and sensing are separated. Further, it is believed that additional information regarding relative location of inclusion and sampling device could be useful in properly positioning a sampling device. Mishra et al (Mishra V, Bouyad H, Schned A, Hartov A, Heaney J, Halter R J. Electrical property sensing biopsy needle for prostate tissue assessment to be published in The Prostate sometime in 2013 have constructed a two-electrode sampling device described above and recorded impedance spectra from 36 ex vivo prostates. The magnitudes of the mean resistive and reactive components were significantly higher in cancer tissues (P<0.05). ROC curves showed that the resistance at 63.09 kHz was optimal for discriminating cancer from benign tissues; this parameter had 75.4% specificity, 76.1% sensitivity, and ROC AUC of 0.779. Similarly, 251.1 kHz was optimal when using the reactance to discriminate cancer from benign tissues; this parameter had a 77.9% specificity, 71.4% sensitivity, and ROC AUC of 0.79. Importantly, they note that “despite the significant differences noted, moderate standard deviations (mean values) were reported” for the impedance values of the different tissue types. This variation is largely thought to be dependent on variable contact impedances associated with a two-electrode approach to tissue sampling. An additional challenge observed with the two-electrode probe with electrodes is located on the tip of each moving needle element is that there is a thin space between these moving elements. Fluids are pulled into this gap through capillary action. These fluids can alter the electrical impedance measurement made between the two needle electrodes. This fluid changes the electrical properties of the insulating layers between the needle electrodes which alters the calibration of the device.

An improved sampling device having four electrodes is illustrated in FIGS. 3, 5, and 6, with an eight-electrode variation illustrated in FIG. 4. This device 200 has a sampling trocar 202 slideably engaged in hollow needle 204, with a sharp point 202A and sampling cutter 202B formed at a first end. Trocar 202 has a handle section 203 formed on a second end of trocar 202. In an embodiment, needle 204 is formed of biocompatible insulating material such as a hard plastic or a ceramic. In an alternative embodiment, needle 204 is formed of a metal, such as stainless steel or non-magnetic brass, with a first biocompatible, electrically insulating, coating (not shown) on its outer surface. The handle section 203 of trocar 202 has an indicator, not shown, indicating a side of trocar 202 on which cutting portion 202B is located.

Formed over the outer surface of needle 204 if needle 204 is nonconductive, or over the first insulating coating if needle 204 is conductive, are four or more conductors 206, 208, 210, 212. Formed over the conductors 206, 208, 210, 212, is an outer electrically insulating, biocompatible, coating 214. A portion of needle 204 for a first distance 216 back from a first end of needle 204 is bare of outer coating 214, exposed portions of the conductors forming electrodes for contacting tissue. The electrode portions of the conductors may be plated with a biocompatible conductive material such as gold. Also bare of outer coating is a portion of needle 204 for a second distance 218 from a second end of needle 204; such that the outer insulating coating covers only a central portion of the needle.

Second end of needle 204 has an orientation key 220 that prevents rotation of the needle in a contactor ring 222, key 220 may take the form of either a notch in needle 204, or a tab formed on needle 204, or may have some other form. Contactor ring 222 has multiple electrical contacts 224, each electrical contact 224 disposed such that it makes contact with a conductor 206, 208, 210, 212 and its associated electrode. Each contact 224 is attached to a wire of a wire bundle or cable 226 for coupling to an impedance measurement apparatus 250.

Impedance measurement apparatus 250 has at least one high frequency alternating-current driver 254 that couple through cable 226 of at least one pair of the electrodes 206, 208, 210, 212, and at least one measurement unit 256 that couples through cable 226 to at least one different pair of the conductors and associated electrodes 206, 208, 210, 212. In a particular embodiment, both current driver 254 and measurement unit 256 couple to cable 226 through an electronic crossbar switching unit 252 that permits coupling of the driver to any pair of the electrodes, and of the measurement unit to any other pair of the electrodes. Both current driver 254 and measurement unit 256 operate under control of a microprocessor 258 executing firmware including machine readable instructions stored in memory 260; microprocessor 258 also drives a display 262 with information derived from impedance measurements. In some embodiments, there is also an additional “subject-ground” or external electrode 264 coupled to the measurement unit, the external electrode 264 coupled through a conductive gel to a common point on the subject's skin. In an embodiment, the impedance measurements are derived by driving a pair of electrodes, such as electrodes 206, 208, while measuring voltages at a different pair of electrodes, such as electrodes 210, 212.

Particular embodiments may have other numbers of electrodes than four, for example an eight-electrode embodiment of the needle is illustrated in cross-section FIG. 4, where additional electrodes 270, 272, 274, 276 are provided.

In an eight-electrode embodiment, electrodes are scanned, by altering a configuration of switching unit 252 under control of processor 258 according to the following table, where electrodes are indicated by reference number in the figures, a “D” indicates electrodes driven, an “M” indicates electrodes measured, the near-field table portion being used to determine tissue impedance of tissue adjacent to the biopsy sampling device, and the distant-field table portion being used in conjunction with near-field results to determine tissue impedance of tissue a little bit further from the biopsy sampling device:

TABLE 1
Rotating Drive and Sense Connection Sequence

| Electrode | 206 | 270 | 212 | 272 | 208 | 274 | 210 | 276 |
|---|---|---|---|---|---|---|---|---|
| Near 1 | D | M | M | D | | | | |
| Near 2 | | D | M | M | D | | | |
| Near 3 | | | D | M | M | D | | |
| Near 4 | | | | D | M | M | D | |
| Near 5 | | | | | D | M | M | D |
| Near 6 | D | | | | | D | M | M |
| Near 7 | M | D | | | | | D | M |
| Near 8 | M | M | D | | | | | D |
| Far 1 | D | M | | M | D | | | |
| Far 2 | | D | M | | M | D | | |
| Far 3 | | | D | M | | M | D | |
| Far 4 | | | | D | M | | M | D |
| Far 5 | D | | | | D | M | | M |
| Far 6 | M | D | | | | D | M | |
| Far 7 | | M | D | | | | D | M |
| Far 8 | M | | M | D | | | | D |

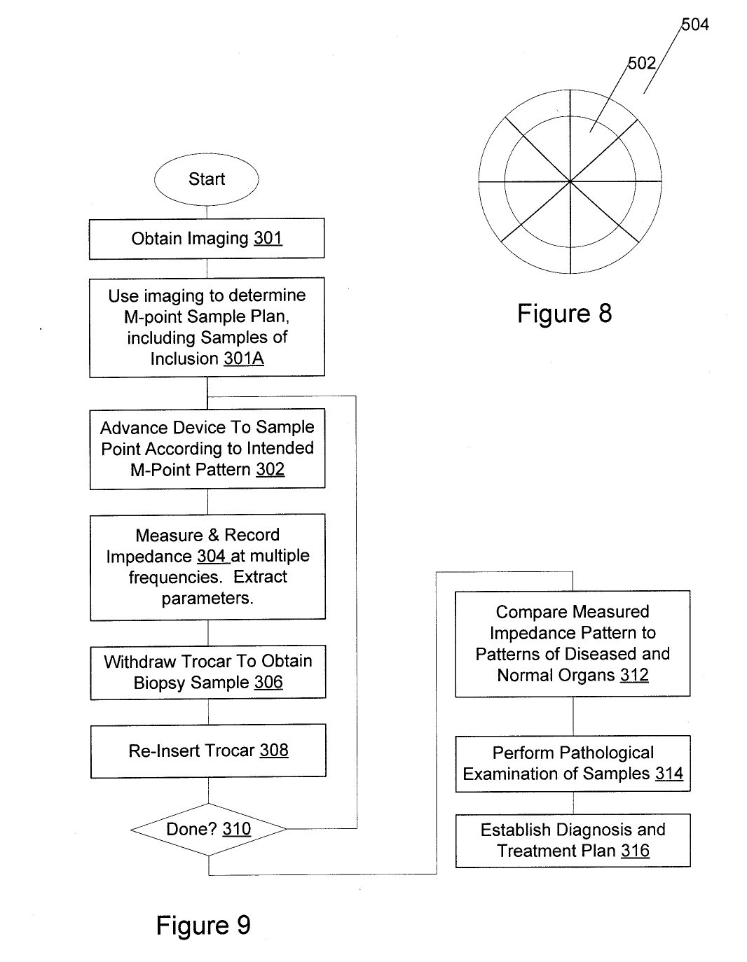

In an embodiment having an eight-electrode tube and near versus far impedance discrimination, tissue impedance determined by processor 258 are displayed in a sectored display, with eight near and eight far segments, as illustrated in FIG. 8. When a particular sector, such as sector 502, has impedance differing from that determined for other sectors, that sector is highlighted with a color different than that displayed for the other sectors.

A system estimates the electrical property distribution around the needle tip using electrical impedance tomography-based algorithms. These algorithms couple together the impedance measurements recorded from all electrode configurations to estimate the spatial distribution of conductivity and permittivity around the needle tip. A map of the electrical properties is provided, which may be displayed as a pie-diagram as illustrated in FIG. 8, to the clinician as a means of representing regions of high or low electrical properties in multiple regions near the needle. Near-probe electrical properties may be displayed as near-probe regions 502.

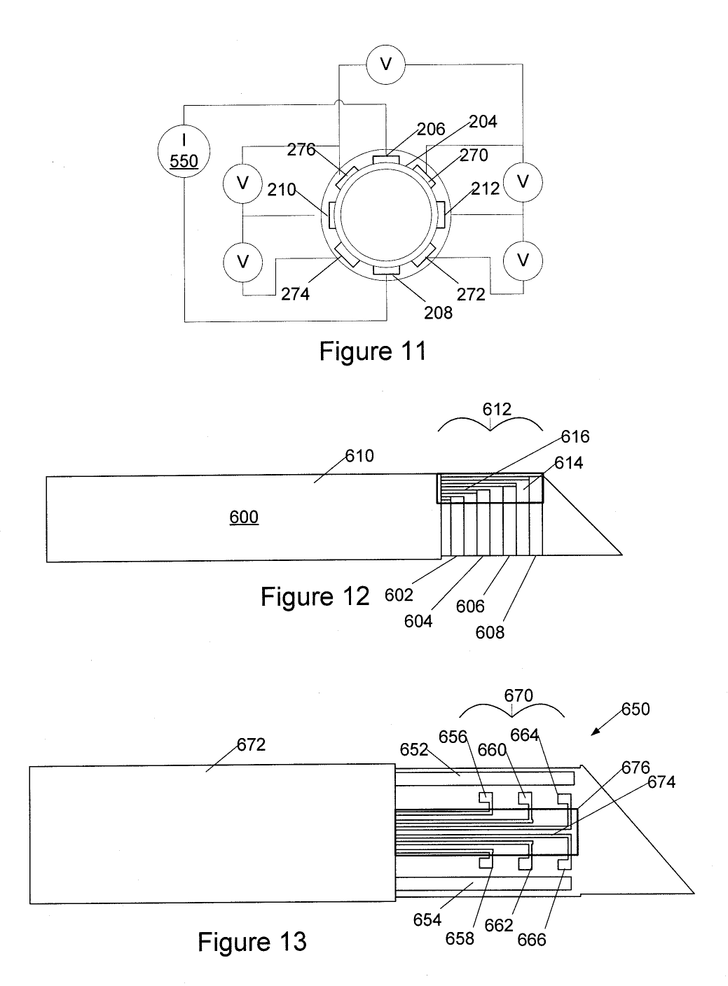

In an alternative embodiment having six or more electrodes, a first pair of electrodes is coupled to a current-driven stimulus source, while voltage measurements are made at more than two other electrodes to estimate electrical properties both as near-probe impedances, and as far-from-probe impedances simultaneously, as illustrated in FIG. 11. Again, switching unit 252 may be used to couple the current stimulation to a different pair of electrodes, and a similar set of measurements taken, to give a fuller picture of near and far tissue impedance.

Far-from-probe properties are displayed to the clinician as outer regions 504 on the pie-diagram as illustrated in FIG. 8. Near-probe and far-from-probe properties are displayed as a color-coded display, with low impedances in a first color, such as blue, and high impedances as a second color, such as red, with intermediate impedances in intermediate colors lying between the first and second colors.

In a first method of operation, as illustrated in FIG. 9, imaging from other modalities (such as Ultrasound, X-ray, CT, or MRI scans, or combinations thereof) is obtained 301. This imaging is used by clinicians to determine the need for biopsy, and to plan 301A multiple M locations, including tumor and near-tumor locations, intended to be sampled during the biopsy procedure. A grid or positioning frame may optionally be used to help guide insertion of the sampling device. During the biopsy procedure, the tip of the sampling device 200 is inserted into the prostate or other organ, and advanced 302 into the prostate, or other organ, to a point at which it is desired to obtain a sample. Guidance of the sampling device may be according to a predetermined pattern, or according to real-time images obtained from the separate imaging modality. In an embodiment, Once at each predetermined sampling point, high frequency impedance characteristics of the tissue are measured 304 by applying a low current, high frequency, stimulus current having at least one, and in an embodiment several, frequencies between 100 hertz (Hz) and 10 MHz by stimulus circuits 254 of the impedance measuring system 201, and measuring voltages developed between other electrodes with the measurement circuit 256 of system 201, these measurements are recorded. In alternative embodiments, frequencies between 100 Hz and 1 MHz are used, and in another embodiment frequencies between 100 Hz and 100 kilohertz (kHz) are used.

In embodiments using multiple frequencies, four multi-frequency based spectral parameters defining the recorded spectrum (σ_{∞}, Δσ, f_{c}, and α) using the Cole-type model are then extracted from the recorded impedance measurements. Other spectral decompositions methods can also be used including Warbug model, discrete component model, constant-phase element models, or general polynomial-based curve fitting models. The sampling needle trocar 202 is then withdrawn 306 to excise and remove a sample from the organ for pathological analysis. Since the stimulus current flows through a radius of about 2½ millimeters around the tip of sampling device 102 several cubic millimeters of the organ are sampled. The measured conductivity, permittivity, and spectral impedance properties give information not just of the sample, but of a region near the sample that may or may not contain possible tumors. If 310 all desired samples have not yet been taken, the trocar 202 is reinserted 308 and the sampling device tip advanced further or otherwise repositioned to obtain additional samples; as an example additional samples might be collected following a predetermined, 12-point, pattern as is often used for prostate biopsy.

Once 310 all desired samples have been taken, the measured pattern of conductivity, permittivity, and spectral parameters, measured within the organ is compared 312 to patterns of conductivity, permittivity, and spectral parameters of both normal and diseased organs. Pathological examination of samples is also performed 314. Both information from the pattern of impedance and spectral parameters, and from the pathological examinations are used to establish 316 a diagnosis and treatment plan. In this method, the impedance and spectral parameter measurements give additional information about tissue characteristics surrounding an analyzed sample that is useful for diagnosis 316, and in particular useful for estimating tumor size and aggressiveness.

The estimated tumor size and aggressiveness is critical to tumor staging; tumor staging in turn is of great interest in devising a treatment plan. In particular, large rapidly growing tumors may require radical prostatectomy, while smaller tumors are more likely to be treated by less invasive techniques such as transurethral resection or active surveillance.

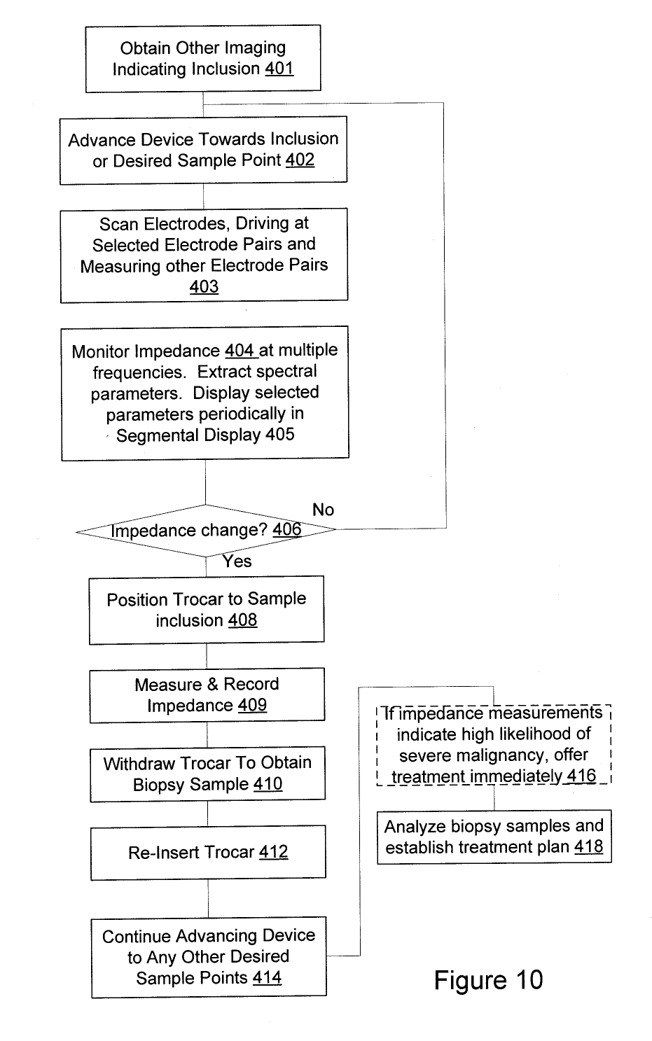

In an alternative method of operation, as illustrated in FIG. 10, impedance changes are used to guide sampling while advancing 402 the sampling device into an organ along a path guided by, or determined according to images obtained 401 by other imaging modalities such as X-ray, CT-scan, MRI-scan, or ultrasound-scan. The images are used, as known in the art of image-guided biopsy, to guide the sampling device 200 towards an inclusion from which a sample is desired. In this method, the impedance characteristics of the tissue are monitored 404 in an area surrounding the probe 202 by scanning 403 the stimulus circuits 254 and voltage sensing circuits 256 across electrodes. This is done by applying a high frequency stimulus current having at least one frequency from the stimulus circuits 254 of the impedance measuring system to a pair of selected electrodes, such as electrodes 206, 208, 210, 212, 270, 272, 274, 276, and measuring voltages developed between two or more selected electrodes other than those being driven with the measurement circuit 256 of impedance measuring system 250.

Monitored 404 measurements are averaged and filtered over a short period of time to avoid artifacts, in embodiments using multiple frequencies the spectral parameters are extracted, and selected impedance measurements and/or spectral parameters for near and far impedance in each direction around the sampling device are displayed to an operator. The measurements are repeated for additional combinations of electrodes according to table 1 and the display, illustrated in FIG. 8, is updated periodically 405. This display can alternatively be an electrical impedance tomogram if that approach is used. When the operator sees a change of impedance in a suspect direction, such as an increase or other change of impedance 406, indicating a suspect inclusion may be near the tip of the sampling device 100, the operator positions the trocar such that its cutter 202B is positioned on a side of the sampling device that is closest to the inclusion and the suspect inclusion is expected to be nearest to the cutter 202B.

Once the sampling device is positioned within the area of suspect impedance, impedance is measured 409 and recorded, and the center trocar 202 of the sampling device is then withdrawn 410 to obtain a biopsy sample of the suspected inclusion. Once the sample is placed in a sample container, the center trocar 202 is reinserted 412 into the sampling device and advancement of the sampling device is then continued towards other locations, such as predetermined locations or locations guided by other imaging methods, within the organ from which samples are to be taken.

In embodiments, both samples according to predetermined locations in the organ and samples according to impedance changes may be taken and submitted for pathological analysis for diagnostic purposes. Information from pathological analysis of the samples, and information from comparing a measured pattern of impedance and spectral parameters at the sampling points to known impedance patterns and spectral parameters of normal and diseased organs, are used in establishing 316, 418 a diagnosis and treatment plan.

In an alternative embodiment of the method, after positioning the sampling slot of the trocar 202 of the device 200 by advancing it into an area of interest in the organ, the outer needle 204 is advanced to excise a sample since cutting by trocar 202 occurs by relative motion of needle and trocar. The trocar 202 is then removed to transfer the sample to a pathology sample container and reinserted into the outer needle 204 before advancing the device to any additional sampling points.

In some embodiments, should impedance measurements indicate a high likelihood of malignancy, treatment may be offered 416 immediately post-biopsy to prevent tissue dislodged by device 200 from forming metastases. Whether or not immediate treatment was offered, the biopsy samples are analyzed and, if necessary, a treatment plan is established 418.

It is expected that multiple-electrode (defined as those having more than two electrodes all located on the sampling device) biopsy sampling devices may have configurations of electrodes other than the multiple electrodes spaced radially around a circumference of the outer needle as illustrated in FIG. 3. In an alternative embodiment, as illustrated in FIG. 12, the outer needle 600 has multiple, part-ring-shaped electrodes, such as electrodes 602, 604, 606, 608, disposed at different distances along a longitudinal axis of the needle, with an outer insulating sleeve 610 that prevents conduction from conductor traces 616 through tissue located proximal to an electrode region 612 near needle 600's tip. Conductor traces 616 are provided and configured to provide electrical continuity between electrodes 602, 604, 606, 608 and contact pad regions at a second end (not shown) of the outer needle. For simplicity, the orientation key and connector contacts pad regions to each conductor trace at the second end of the sampling device configured for contacting contacts 224 are not shown in FIG. 12 or 13. In an embodiment, an insulating patch 614 covers conductor traces 616 near electrode region 612 The outer needle of FIG. 12 is used with an inner trocar 202 similar that of FIG. 3.

In another alternative embodiment, as illustrated in FIG. 13, the outer needle 650 has two, or in a particular embodiment four, longitudinal strip-shaped electrodes 652, 654, with multiple paired patch electrodes 656, 658, 660, 662, 664, 666 between the strip electrodes in an electrode region 670, with an outer insulating sleeve 672 that prevents conduction from conductor traces 674 through tissue located proximal to an electrode region 670 near needle 600's tip. Conductor traces 674 are provided and configured to provide electrical continuity between electrodes 652, 654, 656, 658, 660, 662, 664, 666 and contact pad regions (not shown) electrically coupled to each electrode at a second end (not shown) of the outer needle. For simplicity, the orientation key and connector contacts pad regions to each conductor trace at the second end of the sampling device configured for contacting contacts 224 are not shown in FIG. 12 or 13. In an embodiment, an insulating patch 676 covers conductor traces 674 of paired electrodes near electrode region 670, but leaves the electrodes themselves uncovered The outer needle of FIG. 13 is used with an inner trocar 202 similar that of FIG. 3. Other electrode configurations than those displayed in this document are also possible. The outer needle of FIGS. 12 and 13 is used with a connector ring and electronics similar to that illustrated in FIG. 3.

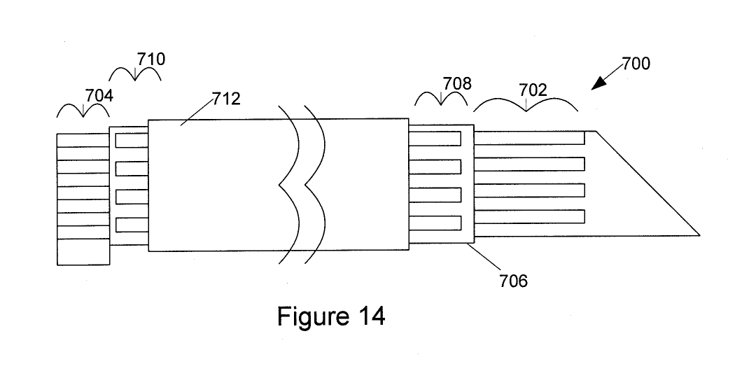

Yet another alternative outer needle 700 is illustrated in FIG. 14. In this embodiment, an inner conductor and electrode layer having multiple electrodes 702 and connector contact pads 704 is formed similarly to the outer needle 202 described with reference to FIGS. 3, 4, 5, and 6 above. An intermediate layer of insulation 706 is formed over a central portion of the inner electrode layer, covering all of the inner electrode conductors except contact pad 704 region and electrode 702 region—for simplicity the conductors are not shown in the figure except in contact pad 704 region and electrode 702 region, however electrical continuity is provided from each contact pad to a corresponding electrode. Over the intermediate layer of insulation 706 is formed a second electrode conductor layer, having second-layer electrodes 708 and second-layer contact pads 710 with electrical continuity is provided from each contact pad to a corresponding electrode in the second electrode conductor layer. An outer layer of insulation 712 is provided over a central region of the second electrode layer to prevent exposure of this layer to tissue except at second-layer electrode region 708. The outer needle 710 is used with measurement apparatus similar to that of FIG. 3 however a second ring of contacts 224 is provided to couple the needle to cable 226 and switch 252. In yet another embodiment, a third, or a particular embodiment a fourth, layer of conductors and electrodes may be formed on outer needle 700.

An outer needle as herein described may be fabricated in several ways. In an embodiment, outer needle 600, 200, 650, is fabricated by forming a printed circuit having electrodes on a thin, insulating, substrate, the substrate is then wrapped about a stainless-steel needle and cemented in place, the substrate becoming the first insulating coating over the conductive needle core previously described with reference to FIG. 3. In an alternative embodiment, a thick first insulating coating is deposited over a stainless steel needle, and a conductive coating deposited over the needle with a hot metal-spray technique. The conductive coating is then selectively removed between desired electrodes using an abrasive wheel to produce linear electrodes as in outer needle 200. In other embodiments, the conductive coating is selectively removed by other photochemical patterning and etching methods. In yet another embodiment, an electrically-conductive ink is printed onto a first insulating coating, and electrodes are formed by electroplating onto the ink.

It is expected that the electrical impedance measurement and monitoring described herein can be added to other biopsy sampling devices that may be known in the art of Medicine.

While the forgoing has been particularly shown and described with reference to particular embodiments thereof, it will be understood by those skilled in the art that various other changes in the form and details may be made without departing from the spirit hereof. It is to be understood that various changes may be made in adapting the description to different embodiments without departing from the broader concepts disclosed herein and comprehended by the claims that follow.

What is claimed is:

1. A biopsy sampling device comprising:
an inner trocar having a sharpened tip and a sampling opening;
an outer needle having a central cavity, the outer needle formed of a material selected from the group consisting of insulators and metal coated with an insulator;
at least a first, a second, a third, and a fourth electrical conductor formed on the outer needle, the conductors forming a first, a second, a third, and a fourth electrode;
an insulating coating formed over at least a central portion of the electrical conductors,
an impedance measuring apparatus coupled to drive current through a first and a second selected electrode of the electrodes, and measure voltages through a third and fourth selected electrode of the electrodes to measure an impedance of tissue adjacent to the electrodes;
an inner trocar slideably engaged within the central cavity of the outer needle such that its sharpened tip and sampling opening may protrude from an end of the outer needle;
wherein the inner trocar is adapted to be removed from the outer needle after positioning it with tip and sampling opening protruding from the end of the outer needle and thereby capture a sample in the sampling opening.

2. The biopsy sampling device of claim 1 further comprising impedance measurement apparatus for measuring an alternating current impedance at at least one frequency between one hundred and ten million hertz, the impedance measurement apparatus coupled to at least one of the needle and trocar.

3. The biopsy sampling device of claim 1 wherein the impedance measurement apparatus measures alternating current impedance at several frequencies between one hundred and one hundred thousand hertz, and computes spectral parameters from the measurements, and displays at least one spectral parameter of impedance to a user.

4. The biopsy sampling device of claim 1 wherein the impedance measurement apparatus is capable of providing stimulus between the third and fourth electrodes while measuring voltages between the first and second electrodes.

5. The biopsy sampling device of claim 4 further comprising a fifth, sixth, seventh, and eighth electrode on the outer needle, and wherein the impedance measuring apparatus is capable of measuring voltages through the seventh and eighth electrodes while driving current through the third and fourth electrodes.

6. The biopsy sampling device of claim 5 wherein the impedance measuring apparatus is capable of measuring voltages through the fifth and sixth electrodes while driving current through the first and second electrodes.

7. The biopsy sampling device of claim 1 further comprising a fifth, sixth, seventh, and eighth electrode on the outer needle, and wherein the impedance measuring apparatus is capable of measuring voltages through the fifth and sixth electrodes while driving current through the first and second electrodes.

8. The biopsy sampling device of claim 1 wherein the electrodes are disposed in a radial pattern on the outer needle.

9. The biopsy sampling device of claim 1 wherein the electrodes are formed in at least two layers on the outer needle.

10. The biopsy sampling device of claim 1 wherein the electrodes are spaced along a longitudinal axis of the outer needle.

11. A method of obtaining biopsy samples from a subject comprising:
advancing a sampling device into an organ while monitoring impedance characteristics between consisting plurality of electrodes formed on an exterior of an outer needle of the sampling device, the outer needle of the sampling device having more than two electrodes;
displaying to an operator on a display at least a first impedance characteristic of organ tissue adjacent the electrodes, the impedance characteristic determined by driving at least one pair of the electrodes while measuring voltages between a second pair of the electrodes, and the impedance characteristic on the display is updated regularly;
upon observing a change of impedance, the operator withdrawing a central trocar of the sampling device to obtain a biopsy sample of an inclusion in the organ.

12. The method of claim 11 wherein the sampling device comprises:
an inner trocar having a sharpened tip and a sampling opening;
the outer needle having a central cavity, the outer needle formed of a material selected from the group consisting of insulators and metal coated with an insulator;
at least a first, a second, a third, and a fourth electrical conductor formed on the outer needle, the conductors forming a first, a second, a third, and a fourth electrode;
an insulating coating formed over at least a central portion of the electrical conductors,
an impedance measuring apparatus configured to determine the impedance characteristic and coupled to drive current through a first and a second selected electrode of the electrodes, while measuring voltages through a third and fourth selected electrode of the electrodes;
the inner trocar slideably engaged within the central cavity of the outer needle such that its sharpened tip and sampling opening protrude from an end of the outer needle;
wherein the inner trocar is adapted to be removed from the outer needle, thereby capturing a sample in the sampling opening.

13. The method of claim 12 further comprising displaying at least a second impedance characteristic determined by driving current through the third and fourth electrodes while measuring voltages through the first and second electrodes.

14. The method of claim 13 wherein the outer needle has at least eight electrodes.

15. The method of claim 13 wherein the first impedance characteristic is a near impedance characteristic representing impedance of tissue near the sampling device, and further comprising:
determining and displaying a far impedance characteristic representing impedance of tissue distal to the sampling device.
